(19) United States
(12) Patent Application Publication
(10) Pub. No.: US 2010/0278946 A1
(43) Pub. Date: Nov. 4, 2010

(54) HERBAL COMPOSITIONS COMPRISING PLANT MATERIAL OF ARTEMISIA HERBA-ALBA (ASSO) ASTERACEAE AND/OR EXTRACTS THEREOF

(76) Inventors: Asher Tyberg, Brooklyn, NY (US); Moshe Ben Arye, Bet El (IL); Aharon Egel-Tal, D.N. Har Hebron (IL)

Correspondence Address: SUGHRUE MION, PLLC, 2100 PENNSYLVANIA AVENUE, N.W., SUITE 800, WASHINGTON, DC, 20037, US

(21) Appl. No.: 12/525,935

(22) PCT Filed: Feb. 14, 2008
(86) PCT No.: PCT/IL08/00196

§ 371 Date: Jan. 12, 2010

(30) Foreign Application Priority Data
Feb. 18, 2007 (IL) 181404

(51) Int. Cl. A61K 36/282; A61P 1/00

(52) U.S. Cl. 424/740

(57) ABSTRACT

A herbal composition including plant material of Artemisia herba-alba (Asso) and/or an extract thereof for alleviating, preventing and/or curing symptoms of any of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting, wherein the herbal composition is substantially free of thujone.

### FIELD OF THE INVENTION

The present invention relates to herbal compositions comprising plant material of Artemisia herba-alba (Asso) and/or an extract thereof for alleviating, preventing and/or curing symptoms of any of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting.

### BACKGROUND OF THE INVENTION

The symptoms and standard treatments for Irritable Bowel Syndrome (IBS), Crohn's Disease, side effects of chemotherapy, colic, abdominal pain, intestinal worms, diarrhea, constipation, gastric ulcer, heartburn, nausea and vomiting are described in detail in the medical literature and handbooks, e.g., The Merck Manual, Merck Research Laboratories (2006) and Physicians' Cancer Chemotherapy Drug Manual, Jones and Bartlett Publishers (2005). The description of these conditions contained therein is incorporated herein by reference.

The ethno botanical and scientific literature contain extensive references to the use of species of Artemisia to alleviate symptoms of various disorders as described herein and in the cited publications and bibliography incorporated herein by reference. However, the use of A. herba-alba or the closely related A. absinthium in its various forms has not achieved wide acceptance for such purposes. It would appear that this is because the alpha- and beta-thujones considered to be the active ingredient of these Artemisia species in their various therapeutic applications are convulsants and act as noncompetitive blockers of the gamma-amino butyric acid (GABA)-gated chloride channel. In many instances, the use of thujone-containing substances in beverages and preparations for human consumption is restricted by law. According to the Encyclopedia Britannica 2007 Deluxe Edition, Chicago, absinthe came to be considered dangerous to one's health because it appeared to cause convulsions, hallucinations, mental deterioration, and psychoses, these symptoms evidently being caused by thujone. Apparently, this is why absinthe manufacture was prohibited in Switzerland in 1908, in France in 1915, and eventually in many other countries.

The following references and selected quotations cited therefrom provide a comprehensive background for the present invention.

### REFERENCES

Artemisia Herba-Alba—General Information
Handbook of Arabian Medicinal Plants by Shahina A Ghazanfar-CRC Press, 1994, page 40
Handbook of Phytochemical Constituents of Grass Herbs and Other Economic Plants by James A Duke-CRC Press-2000 and others

Uses in Humans
Felter H W, Lloyd J U, King's American Dispensatory, Ohio Valley Co., Cincinnati, 18th ed. 1898
University of Michigan, A Database of Foods, Drugs, Dyes and Fibers of Native American Peoples, Derived from Plants, Native American Ethno botany, online database, 2006
Plants of the Bible, Harold N. Moldenke, Alma L. Moldenke, Ronald Press Company, New York, 1952, page 48
Dr. Duke's Phytochemical and Ethno botanical Databases, USDA Agricultural Research Service online database. Ethno botanical uses-Artemisia herba-alba 2006
Friedman J, Yaniv Z, Dafni A, Palewitch D., J. Ethnopharmacol. 1986 June; 16(2-3):275-87 A preliminary classification of the healing potential of medicinal plants, based on a rational analysis of an ethno pharmacological field survey among Bedouins
Sahara and Sudan Vol 1 Fezzan and Tibesti by Gustav Nachtigal-C. Hurst & Co. Publishers, 1975, page 140
Handbook of Arabian Medicinal Plants by Shahina A Ghazanfar-CRC Press, 1994, page 40
Non-Wood Forest Products in the Near East: a Regional and National Overview by Amal Sabra and Sven Walter and Tunisia Herbal and Medicinal Plants Project (HMPP), Supervision Mission, Jun. 8-14, 2003, Aide Memoire, No. 2
Institut de Physique Nucléaire-Artemisia herba alba, online database, 2006
Plants For A Future, A Resource and Information Centre for Edible and Other Useful Plants-Artemisia, absinthium, England and Wales. Charity No. 1057719, Company No. 3204567, online database, 2004
Blumenthal M, Busse W, Goldberg A, Gruenwald J, Hall T, Riggins C, Rister R, The Complete German Commission E Monographs, Therapeutic Guide to Herbal Medicines-Absinthial Herba, American Botanical Society, Austin, Tex., 1998
Yashphe J, Segal R, Breuer A, Erdreich-Naftali G h. J Pharm Sci (1979 July) 68(7):924-5 reported on antibacterial activity of A. herba-alba and concluded that only its essential oil was active against some Gram-positive and Gram-negative bacteria.

Thujone as an Active Ingredient:
Pato [symbol] ka J, Plucarb B, Pharmacology and toxicology of absinthe, Journal of Applied Biomedicine, 2003; 1:199-205
   Quote: “Thujone is probably the most important biologically active compound of absinthe . . . . Alpha-thujone is the principal active ingredient of wormwood oil and toxic principle in absinthe (Arnold 1988).”
Harrison K, 3 dchem-Absinthe, online database of Dr. Karl Harrison, Department of Chemistry, University of Oxford
   Quote: “The presumed active ingredient in wormwood's oils, alpha-thujone . . . ”
Hold K M, Sirisoma N S, Casida J E, Detoxification of alpha- and beta-Thujones (the active ingredients of absinthe): site specificity and species differences in cytochrome P450 oxidation in vitro and in vivo, Chem Res Toxicol. 2001 May; 14(5):589-95
   Quote: “Alpha- and beta-thujones are active ingredients in the liqueur absinthe and in herbal medicines and seasonings for food and drinks. Our earlier study established that they are convulsants and have insecticidal activity, acting as noncompetitive blockers of the gamma-amino butyric acid (GABA)-gated chloride channel, and identified 7-hydroxy-alpha-thujone as the major metabolite and 4-hydroxy-alpha- and beta-thujones and 7, 8-dehydro-alpha-thujone as minor metabolites in the mouse liver microsome-NADPH system. We report here unexpected site specificity and species differences in the metabolism of the thujone diastereomers in mouse, rat, and human liver microsomes and human recombinant P450 (P450 3A4), in orally treated mice and rats, and in Drosophila melanogaster . . . ”
Sirisoma N S, Hold K M, Casida J, Alpha- and beta-Thujones (herbal medicines and food additives): synthesis and analysis of hydroxy and dehydro metabolites. J Agric Food Chem. 2001 April; 49(4):1915-21
   Quote: “Essential oils containing alpha- and beta-thujones are important herbal medicines and food additives. The thujone diastereomers are rapidly metabolized convulsants acting as noncompetitive blockers of the gamma-aminobutyric acid-gated chloride channel.”
Chiasson H L N, Belanger A, Bostanian N, Vincent C, Poliquin A, Acaricidal Properties of Artemisia absinthium and Tanacetum vulgare (Asteraceae) Essential Oils Obtained by Three Methods of Extraction, J. Econ. Entomol. 94(1):167-71 (2001)
   Quote: “Chemical analysis of the T. vulgare extracts indicated that b-thujone is by far the major compound of the oil (>87.6%) and probably contributes significantly to the acaricidal activity of the oil.”

Detrimental Side Effects of Absinthe
Opinion of the Scientific Committee on Food on thujone, SCF/CS/FLAV/FLAVOUR/23 ADD2 Final 6 Feb. 2003, European Commission Health & Consumer Protection Directorate-General
Arnold W N, Vincent Van Gogh and the Thujone Connection, JAMA 1988; 260:3042-3044

Side Effects of Absinthe Attributed to Thujone
National Toxicology Program Fact Sheet—Herbal Medicines, 2003, Dr. Tom Burka, National Institute of Environmental Health Sciences (NIEHS), U.S.A.
   Quote: “Thujone: Terpenoid found in a variety of herbs, including sage and tansy, and in high concentrations in wormwood. Suspected as the causative toxic agent associated with drinking absinthe, a liqueur flavored with wormwood extract.” Hold K M, Sirisoma N S, Ikeda T, Narahashi T, Casida J E
Hold K M, Sirisoma N S, Ikeda T, Narahashi T, Casida J E Alpha-thujone (the active component of absinthe): gamma-aminobutyric acid type A receptor modulation and metabolic detoxification, Proc Natl Acad Sci USA. 2000 Apr. 11; 97(8):3826-31.
   Quote: “Alpha-thujone is the toxic agent in absinthe, a liqueur popular in the 19th and early 20th centuries that has adverse health effects. It is also the active ingredient of wormwood oil and some other herbal medicines and is reported to have antinociceptive, insecticidal, and anthelmintic activity. This study elucidates the mechanism of alpha-thujone neurotoxicity and identifies its major metabolites and their role in the poisoning process. Four observations establish that alpha-thujone is a modulator of the gamma-aminobutyric acid (GABA) type A receptor . . . . Third, alpha-thujone is a competitive inhibitor of [(3)H]ethynylbicycloorthobenzoate binding to mouse brain membranes. Most definitively, GABA-induced peak currents in rat dorsal root ganglion neurons are suppressed by alpha-thujone . . . .”

Laws and Regulations Limiting Thujone's use
European Union: Annex II of Directive 88/388/EEC (EEC, 1988), European Commission, Health & Consumer Protection Directorate—General Directorate C —Scientific Opinions, C2—Management of scientific committees; scientific co-operation and networks, Scientific Committee on Food, SCF/CS/FLAV/FLAVOUR/23 ADD2 Final 6 Feb. 2003 Opinion of the Scientific Committee on Food on Thujone (expressed on 2 Dec. 2002)
   Quote: “on flavourings sets the following maximum levels for thujone ( [symbol] and β) in foodstuffs and beverages to which flavourings or other food ingredients with flavouring properties have been added: 0.5 mg/kg in foodstuffs and beverages [with the exception of 5 mg/kg in alcoholic beverages with not more than 25% volume of alcohol; 10 mg/kg in alcoholic beverages with more than 25% volume of alcohol; 25 mg/kg in foodstuffs containing preparations based on sage; and 35 mg/kg in bitters] Thujone may not be added as such to food.”
Codex Alimentarius, 1979, the Codex Committee on Food Additives Paraphrase:
   Paraphrase: recommended restricting the use of thujone (α and β) to the following maximum levels in final products for consumption: 0.5 mg/kg in food and beverages; 5 mg/kg in alcoholic beverages containing <25% alcohol; 10 mg/kg in alcoholic beverages containing >25% alcohol; 35 mg/kg in bitters (Codex Alimentarius Commission 1979).
U.S. Code of Federal Regulations Title 21, Volume 3, 21CFR172.510.
   Paraphrase: Thujone is not authorized for use as a flavouring substance in the USA

Thujone Free Varieties
Juteau F, Jerkovic I, Masotti V, Milos M, Mastelic J, Bessiere J M, Viano J. Composition and antimicrobial activity of the essential oil of Artemisia absinthium from Croatia and France, Planta Med 2003; 69:158-161.
   Quote: “As they contain no thujone, antimicrobial screening was performed on samples of French origin and showed that A. absinthium oil inhibited the growth of both tested yeasts . . . ”
Pato\check{c}ka J, Plucarb B, Pharmacology and toxicology of absinthe, Journal of Applied Biomedicine, 2003; 1:199-205.
   Quote: “despite being smoked for its psychoactive effects, an assay of Artemisia nilagirica oil found it contained less than one percent total thujones.”

Commercial Uses
Hager's Handbuch Der Pharmazeutischen Praxis, Ed. Frerichs G., Springer-Verlag, Germany, 1949, pp. 582-584
Huertas Orgánicas-Artemisia absynthium, online database, 2006
Israeli Health Ministries, Registry of Medicinal Plants for pharmaceutical use, 1982
Remington's Practice of Pharmacy, E W Martin, E F Cook, Mack, USA, 1956, page 988
Sahara and Sudan Vol 2 Kawar, Bornu, Kanem, Borku, Ennedi by Gustav Nachtigal—C. Hurst & Co. Publishers, 1980, page 211
An Ottoman Century: The District of Jerusalem in the 1600s by Dror Ze'evi —SUNY. Press 1996, page 106
375 Essential Oils and Hydrosols by Jeanne Rose, Frog Ltd, 1999

Contraindications and Precautions
Ciganda C, Laborde, A. Herbal Infusions Used for Induced Abortion, J Toxicol Clin Toxicol, 2003; 41(3):235-9

Drug Interactions
University of Michigan Health System Drug Information Service, Selected Herb-Drug Interactions, online database, 2006

Overdosage-Effects
PDRHealth, Physicians' Desk Reference, Thomson Healthcare, 2006
Lenz K L, Alternative Medicines, published in Swarbrick J, and Boylan J, Eds., Encyclopedia of Pharmaceutical Technology, 2nd ed., Marcel Dekker, Inc., 2002
Woolf A, Essential Oil Poisoning, Journal of Toxicology—Clinical Toxicology; 37(6):721-727
O'Neil C K and Fetrow, C W, Plants as Drugs, published in Swarbrick J, and Boylan J, Eds., Encyclopedia of Pharmaceutical Technology, 2nd ed., Marcel Dekker, Inc., 2002, vol. 3, pp. 2214-2225
Leung A Y, Foster S. Encyclopedia of Common Natural Ingredients Used in Food, Drugs, and Cosmetics, 2d ed. New York: John Wiley & Sons, 1996, pp. 1-3

Pharmacological Data
Ordóñez M G, Idavoy D T, Pol L M, Validación del uso tradicional de plantas medicinales cultivadas en Cuba, Rev Cubana Plant Med, 2001; 2:48-51
Shirazi M H, Fazeli M R, Sultan Dalal M M, Eshraghi S, Jamalifar H Alamulhoda E, A comparative study on the antimicrobial effect of some medicinal herbal extracts and selective antibiotics against the clinical isolates of helicobactor pylori, J. of Medicinal Plants (7), Iranian Academic Center for Education, Culture & Research (ACECR)

Clinical Studies
Thompson-Coon J, Ernst E. Systematic review: herbal medicinal products for non-ulcer dyspepsia. Aliment pharmacol Ther 2002; 16: 1689-99

Toxicology
al-Khazraji S M, al-Shamaony L A, Twaij H A entitled: Hypoglycaemic effect of Artemisia herba alba. I. Effect of different parts and influence of the solvent on hypoglycaemic activity. In: J Ethnopharmacol (1993 December); 40(3):163-6
WHO Food Additives Series 16—Thujone, FAS 16-JECFA 25/227, International Programme on Chemical Safety, INCHEM, 1981
Khattak S G, Gilani S N, Ikram M. Antipyretic studies on some indigenous Pakistani medicinal plants. J Ethnopharmacol 1985; 14:45-51
Opinion of the Scientific Committee on Food on Thujone, SCF/CS/FLAV/FLAVOUR/23 ADD2 Final 6 Feb. 2003, European Commission Health & Consumer Protection Directorate-General
Pato\check{c}ka J, Plucarb B, Pharmacology and toxicology of absinthe, Journal of Applied Biomedicine, 2003; 1:199-205
   Quote: “Alpha-thujone . . . is also the active ingredient of wormwood oil and some other herbal medicines and is reported to have antinociceptive, insecticidal, and anthelmintic activity. The content of beta-thujone often exceeds that of alpha-thujone depending on the plant source, but the beta-diastereomer is generally of lower toxicity. The plant Artemesia absinthium and wormwood oil have insecticidal properties (Grainge and Ahmed, 1988), and alpha-thujone was one of the two most toxic monoterpenoids tested against western corn rootworm larvae (Lee et al. 1997) . . . . The toxic effects of alpha-thujone in mammals are well established but the mode of neurotoxic action is not fully known (Bonkovsky et al. 1992). Alpha-thujone is neurotoxic in rats (Millet et al. 1981). Furthermore, it is not even clear that thujone is present in sufficient quantities to play a role in the absinthe intoxication of humans . . . . However, it is possible that thujone accumulates in the body and plays a role in the psychoactivity and toxicity of chronic absinthe use. The i.p. LD50 of alpha-thujone in mice is about 45 mg/kg and the toxicity curve is very sheer. With a dose of 30 mg/kg no death was observed and with a dose of 60 mg/kg 100% mortality was recorded Mice at the higher dose undergo a tonic convulsion leading to death within 1 min whereas at 30-45 mg/kg they exhibit tail-raising within the first 2 min, followed by flexion of the trunk and clonic activity of the forelimbs, progressing to generalized and protracted tonic/clonic convulsions that ultimately result in death or recovery. The only proven effect of thujone, however, is its toxicity to the brain. Thujone may play a role in absinthe, but the evidence is not conclusive. Finally, it should be noted that by focusing on one component of wormwood oil, we ignore the many other poorly characterized compounds in wormwood and absinthe's other herbal ingredients which may play some role in absinthe's intoxicating and toxic effects. Intraperitoneal administration of diazepam or phenobarbital 15 min before alpha-thujone at 100 mg/kg results in almost all of the mice surviving this otherwise lethal dose . . . . Drosophila of the dieldrin-resistant (Rdl) strain are also resistant to alpha-thujone. Alpha-thujone modulates the GABAA receptor based on four observations. Comparison with picrotoxinin, the classical GABAA receptor antagonist, revealed similar poisoning signs and in both cases alleviation of toxicity by diazepam, phenobarbital, and ethanol (Kulkami et al. 1990, Enna et al. 1997). Most importantly, electrophysiological studies establish that in dorsal root ganglion neurons alpha-thujone is a reversible modulator of the GABAA receptor. Alpha-thujone is a competitive inhibitor of ethynylbicycloorthobenzoate (EBOB) binding, i.e., of the noncompetitive blocker site of the GABA gated chloride channel (Ratra et al. 2001). Absinthe and wormwood oil contain not only alpha-thujone as their purported active ingredient but also many other candidate toxicants, including beta-thujone and ethanol in the case of absinthe, but alpha-thujone is the most common candidate on the neurotoxicity principle. Beta-thujone is less toxic than alpha-thujone to mice (Rice and Wilson, 1976) and Drosophila and in addition is 2.3-fold less potent in the [3H]EBOB assay. Current low levels of alpha- and beta-thujone in absinthe are of much less toxicological concern than the ethanol content (Strang et al. 1999). Another possibility exists that the toxic effect of thujone is realized by means of its metabolites. Alpha-thujone as other monoterpenes is easily metabolized. The single report on metabolism identifies thujol and neothujol probably as conjugates in the urine of thujone-treated rabbits (Ishida et al. 1989). Hold et al. (2001) found enzymatic reduction of alpha-thujone to thujol and neothujol in low yield by rabbit but not mouse liver cytosol with NADPH. The mouse liver microsomal P450 system rapidly converts alpha-thujone to 7-hydroxy-alpha-thujone as the major metabolite and the diastereomers of 4-hydroxythujon and some other hydroxythujones as minor metabolites. The various hydroxythujones probably are not the terminal metabolites because they are expected to undergo conjugation and excretion. However, the presence of hydroxythujones in the brain suggests their potential importance in the neurotoxicity. From this point of view there are two principal candidate toxicants: alpha-thujone and its 7-hydroxy metabolite. The 7-hydroxy compound was found in the brain at much higher levels than the parent alpha-thujone, suggesting possible conversion in situ, but this oxidation was not observed on incubation of alpha-thujone with brain microsomes and NADPH. Alpha-thujone compared with 7-hydroxy-alpha-thujone is 56-fold more potent in the [3H]EBOB binding assay and much more toxic to mice and houseflies. It appears that all of the metabolites are detoxification products, i.e., less toxic than alpha-thujone. Importantly, alpha-thujone appears at much lower levels and is less persistent than 7-hydroxy-alpha-thujone. At severely toxic alpha-thujone doses (40-60 mg/kg) the levels in the brain at 30 min after administration of alpha-thujone were 0.3-1.0 ppm for alpha-thujone and 1.5-8.4 ppm for 7-hydroxyalpha-thujone with much higher levels (11 and 29 ppm for alpha-thujone and 7-hydroxy-alpha-thujone, respectively) at 2.5 min when the poisoning signs are most intense. The minor hydroxythujone metabolites are detectable only up to 20 min after the 50 mg/kg alpha-thujone dose. The discovery of alpha-thujone and its metabolite in brain suggests that either one or both may contribute to the toxic manifestations. Unfortunately, the metabolism of alpha-thujone in humans is not known.”

The present invention relates to substantially thujone free preparations of Artemisia herba-alba (Asso) possessing effective therapeutic properties.

### SUMMARY OF THE INVENTION

There are provided here, in accordance with embodiments of the present invention, a herbal composition comprising plant material of Artemisia herba-alba (Asso) and/or an extract thereof for alleviating, preventing and/or curing symptoms of any of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting, wherein the herbal composition is substantially free of thujone.

In accordance with some embodiments of the present invention, a unit dosage form of the herbal composition is provided. This dosage form may comprise 0.2 g to 1.2 g of dry plant material of Artemisia herba-alba (Asso) or an extract thereof. In some embodiments, the herbal composition may be an aqueous extract. In some embodiments, the herbal composition may be adapted for oral consumption or administration. It may be in the form of a hot or cold infusion (aqueous extract), capsule or a pill. In accordance with some embodiments, the herbal composition, when in the form of an aqueous extract, may be taken as a tea.

There is also provided, in accordance with the embodiments of the present invention, the use of a herbal composition in Artemisia herba-alba (Asso) for the preparation of compositions for alleviating, preventing and/or curing symptoms of any of the following: Irritable Bowel Syndrome (IBS), Crohn's Disease, side effects of chemotherapy, colic, abdominal pain, intestinal worms, diarrhea, constipation, gastric ulcer, heartburn, nausea and vomiting.

The present invention also relates to a method for alleviating, preventing and/or curing symptoms of any of the following: Irritable Bowel Syndrome (IBS), Crohn's Disease, side effects of chemotherapy, colic, abdominal pain, intestinal worms, diarrhea, constipation, gastric ulcer, heartburn, nausea and vomiting by administering to a subject suffering from any of the aforementioned, an effective dose of the herbal compositions described herein.

### DETAILED DESCRIPTION OF EMBODIMENTS OF THE INVENTION

The particular phenotype and genotype of Artemisia herba-alba (Asso) which was used in the experiments described below was grown in Israel between Beer Sheva and Mitzpeh Ramon as well as in the area of the Judean Desert and Hebron. This genotype of “Artemisia herba-alba” does not contain alpha- or beta-thujone, a molecular component of wormwood, cedar leaf, sage and tansy, the use of which is restricted in the US and Europe.

The plant material for the formulations used in the experiments described below, were harvested in the summer season when the plant bears small round leaves, after they ripened. At that time they range from light gold to brown (generally golden-brown). This happens from approximately May to June, although it is possible to find them before and afterwards. The plant is picked during blossoming and is put through a process of drying in air in a well-ventilated area in the dark.

The main pharmacologically active constituents in Artemisia herba-alba are mainly located in the round leaves that drop off during the drying process. The plant is preferably added to water in the form in which it grows, without being ground up, and with most of the round leaves (which contain the majority of the active substance) on the branches of the plant, or alternatively, in the form that they are in when gathered from the bottom of the bin in which they were dried.

Preparation of a Composition for Administration

When fresh and dry, the plant was processed by washing it with cold water and transferring it into previously boiled water. The plant material was added to the boiled water for 10 minutes only after boiling was stopped and the water's temperature had decreased to 98 degrees Celsius. The water extract thus obtained was analyzed using GC/MS and found to be free of thujones. The plant was then taken out of the water and the liquid was processed as required into various dosage forms—an infusion, syrup, pills and capsules. These dosage forms may be preserved by the addition of sugar to the syrup form and a fungus inhibiting agent and a regular preservative in the pill and possible capsule form. There is another option of freeze-drying the liquid and then transforming it to one of the forms of the composition. The preparation was done with the plant material being whole and not ground up. Several additional, mildly-active, plants [such as Louisa, Melissa Officinalis, Mint] may be added.

The dosage administered to the subjects was based on between 0.2 g to 1.2 g of dry plant material for a single dose of the composition (about 30-180 ml of water). As the liquid is bitter, adding sugar is possible but not necessary. The plant material used was tested using GC/MS and shown to be substantially free of thujones.

### EXAMPLES

### Example 1

### Irritable Bowel Syndrome (IBS)

A group of 30 subjects diagnosed to be suffering from symptoms of IBS for a period between a few weeks and 20 years were given the composition. Twenty-four subjects received the tea form and six received the composition in the form of a capsule. Each subject received a dose based on between 0.2 g to 1.2 g of dry plant material that was administered once a day for a period of 10 days. The group consisted of males and female adults selected randomly. Substantial relief, as measured by patients' responses to questions about how they felt, was noted after 1-15 days in the majority of the subjects. 18 were cured completely. Eight reported some relief. Four did not feel any relief.

The disappearance of symptoms for one year is considered a complete cure.

### Example 2

### Crohn's Disease

A group of six subjects diagnosed to be suffering from symptoms of Crohn's Disease for a period between six months and two years, were given a daily dose of the composition. The composition was administered as a tea, with a dosage based on between 0.2 g and 1.2 g of dry plant material that was given once a day for a period of 30 days. The group consisted of male and female adults selected randomly. Substantial relief, as measured by patients' responses to questions about how they felt, was noted after one day in most of the subjects. Two were cured completely and felt relief for the next six months.

The disappearance of the symptoms is considered a complete cure.

### Example 3

### Side Effects of Chemotherapy

Two subjects diagnosed to be suffering from upset stomachs, loss of appetite, nausea and vomiting as a result of being subjected to chemotherapy, were given a dose of the composition administered as a tea. The dosage was based on 0.5 g of dry plant material that was administered twice a day. Both subjects were female adults selected randomly. Substantial relief, as measured by patients' responses to questions about how they felt, was noted after one day in both subjects. The dosage was given, and relief felt, as long as the patients were undergoing chemotherapy

### Example 4

### Colic

A group of 25 subjects diagnosed to be suffering from symptoms of colic were given a dose of the composition. The composition was administered as a tea with a dosage based on 0.2 g of dry plant material that was given twice a day until the symptoms were gone. The group consisted of babies two to six months old, males and females being selected randomly. Substantial relief, as measured by the parents' responses to questions about their child's behavior, was noted after one day in most of the subjects. 14 were cured completely. Three felt some relief.

The disappearance of the symptoms is considered a complete cure.

### Example 5

### Abdominal Pain

Sixty subjects suffering from abdominal pain were given a dose of the composition administered as a tea. The dosage was based on 0.5 g of dry plant material, that was given twice a day. 55 subjects, as measured by patients' responses to questions about how they felt, noted immediate relief. Five felt no relief. The termination of the pain is considered as cured.

### Example 6

### Intestinal Worms

A group of 30 people suffering from intestinal worms were given a dose of the composition administered as a tea. The dosage was based on 0.5 g of dry plant material that was given twice a day. 26 subjects, as measured by the disappearance of intestinal worms, noted relief within two days and were cured. Four were not assisted by the tea.

The disappearance of intestinal worms is considered a complete cure.

### Example 7

### Diarrhea

A hundred subjects suffering from diarrhea for a period between three days and two weeks, received the composition, 60 in tea form, 20 as capsules, 10 in the form of melting tablets, and 10 as a syrup. The dosage was based on between 0.2 g to 1.2 g of dry plant material that was administered twice a day for a period of one week. Relief, as measured by patients' responses to questions about how they felt, was noted after one day by 90 subjects. 10 subjects did not note any relief.

The disappearance of the symptoms is considered a complete cure.

### Example 8

### Constipation

A group of 30 people suffering from constipation for a period between one month and several years, received the composition, 25 as a tea and five in a form of melting tablets. The dosage was based on between 0.5 g to 1.2 g of dry plant material that was administered twice a day for a period of three days to one week. Relief, quantified by having a bowel movement once a day, was felt by 22 subjects. Eight subjects felt no relief.

The disappearance of the symptoms is considered a complete cure.

### Example 9

### Gastric Ulcer

Six subjects, suffering from gastric ulcers for a period between two months and a year, received the composition as a tea. The dosage was based on 0.5 g of dry plant material, that was administered twice daily. Significant relief, as measured by patients' responses to questions about how they felt, was felt by five out of the six subjects, three being cured within three months. One subject felt no relief.

The disappearance of the symptoms for a year is considered a complete cure.

### Example 10

### Heartburn

Twenty subjects suffering from heartburn for a period between one month and six months received the composition. 12 people received the composition as a tea, four as a tablet and four as a capsule. Dosage was based on 0.5 g of dry plant material that was given once a day. Immediate relief, as measured by patients' responses to questions about how they felt, was felt by 12 people. Three subjects reported relief after two to three days. The rest did not report any relief.

The disappearance of the symptoms is an indication of a complete cure.

### Example 11

### Nausea and Vomiting

A group of 15 subjects, suffering from nausea and vomiting for a period between three days and two weeks, received the composition as a tea. The dosage was based on between 0.5 g to 1.2 g of dry plant material that was administered once or twice a day for the duration of the above symptoms which was between two days to three months. Relief, as measured by patient responses to questions about how they felt, was noted immediately or at the most, three days.

The disappearance of the symptoms is considered a complete cure.

This application attempts to set forth the most practical way of carrying out embodiments of the invention described herein. Even so, those of ordinary skill in the art will understand that many modifications may be made without deviating from the scope or spirit of the invention. The scope of the invention described herein and the incorporated references, is to be accorded the broadest interpretation of the appended claims, so as to encompass all equivalent methods and products.

1. A herbal composition comprising at least one of (a) plant material of Artemisia herba-alba (Asso) or (b) an extract thereof for alleviating, preventing and/or curing symptoms of any of the following conditions:
Irritable Bowel Syndrome (IBS), Crohn's Disease, side effects of chemotherapy, colic, abdominal pain, intestinal worms, diarrhea, constipation, gastric ulcer, heartburn, nausea and vomiting
wherein the herbal composition is substantially free of thujone.

2. A composition of claim 1 which is in unit dosage form comprising 0.2 g to 1.2 g based on dry plant material of said at least one of (a) plant material or (b) extract thereof, wherein said plant material is portions of Artemisia herba-alba (Asso) and said extract thereof is an extract of dry plant material of Artemisia herba-alba (Asso).

3. A composition according to claim 1 wherein the plant material is ripe small round leaves.

4. The composition according to claim 1, wherein the extract is an aqueous extract.

5. The composition according to claim 1, wherein the composition is orally consumable or administrable.

6. The composition according to claim 1, wherein the composition is in the form of an infusion (tea), capsule or a pill.

7. The composition according to claim 4, wherein the aqueous extract is in the form of a tea or a syrup.

8. The use of a herbal composition in accordance with claim 1 for the preparation of a composition for alleviating, preventing and/or curing symptoms of one or more of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting.

9. The composition in accordance with claim 1 for alleviating, preventing and/or curing symptoms of one or more of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting.

10. A method for alleviating, preventing and/or curing symptoms of one or more of the following conditions: Irritable Bowel Syndrome (IBS), Crohn's Disease, side effects of chemotherapy, colic, abdominal pain, intestinal worms, diarrhea, constipation, gastric ulcer, heartburn, nausea and vomiting, by administering to a subject suffering therefrom an effective dose of the herbal composition in accordance with claim 1.

11. The method of claim 10 wherein the herbal composition is in a unit dosage form comprising 0.2 g to 1.2 g based on dry plant material of said at least one of (a) plant material or (b) an extract thereof, wherein said plant material is portions of Artemisia herba-alba (Asso) and said extract thereof is an extract of dry plant material of Artemisia herba-alba (Asso).

12. The method according to claim 10 wherein the plant material is ripe small round leaves.

13. The method according to claim 10, wherein the extract is an aqueous extract.

14. The method according to claim 10, wherein the composition is orally consumable or administrable.

15. The method according to claim 10, wherein the composition is in the form of an infusion (tea), capsule or a pill.

16. The method according to claim 13, wherein the aqueous extract is in the form of a tea or a syrup.
